(19) United States
(12) Patent Application Publication
(10) Pub. No.: US 2008/0249402 A1
(43) Pub. Date: Oct. 9, 2008

(54) System or Synchronised Playback of Video Image Clips

(75) Inventors: Michael Szucs, Redmond, WA (US); Stephanie Gethmann, Kirland, WA (US)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS, N.V., EINDHOVEN (NL)

(21) Appl. No.: 11/575,673

(22) PCT Filed: Sep. 27, 2005
(86) PCT No.: PCT/IB05/53186

§ 371 Date: Feb. 14, 2008

(30) Foreign Application Priority Data
Sep. 29, 2004 (US) 60614385

(51) Int. Cl. A61B 8/08

(52) U.S. Cl. 600/437

(57) ABSTRACT

The present invention relates to an ultrasound image review system which synchronises, for simultaneous display, two or more ultrasound image clips (A, B, C) captured in respect of, for example, the cardiac region of a patient. Each clip (A, B, C) is divided into frame sets (200, 300, 400), wherein each frame set (200, 300, 400) corresponds to a physiological cycle, such as the cardiac cycle (H), of the patient. Frame sets (200, 300, 400) of the clips (A, B, C) corresponding to each currently-viewed cardiac cycle (H) are then synchronised, such that parameters, such as heart case and differing frame counts between clips, are taken into account. A shorter clip can wrap round to the beginning and remain synchronised to a larger clip, while maintaining continuous playback of both clips. Synchronised clips start and end simultaneously.

### FIELD OF THE INVENTION

The present invention relates to relates to a system for synchronised playback of video image clips and, more particularly but not necessarily exclusively, to a system for synchronised playback of video image clips of varying lengths obtained by means of an ultrasonic diagnostic imaging system, such as an echocardiographic system.

### BACKGROUND OF THE INVENTION

One of the advantages that diagnostic ultrasound has had over many other diagnostic imaging modalities is the ability to produce realtime images. This advantage has been especially significant in echocardiography where the physiology of a continually moving organ, i.e. the heart, is the subject of study. Realtime imaging has been a virtual necessity in echocardiography, as compared with abdominal and obstetrical applications where the tissues and organs being studied are stationary and may be readily examined by static imaging. Echocardiologists, like other practitioners of diagnostic ultrasound, make records of their ultrasound examinations for subsequent diagnosis, review and comparison. Since echocardiographic studies use realtime ultrasonic imaging, they have conventionally been recorded on videotape with a VCR, rather than being recorded statically on film or as photographic prints. A VCR has thus been an essential accessory for an echocardiographic system for many years.

More recently, ultrasound image clips (i.e. a series of ultrasound image frames) have been stored in a digital format for playback on an ultrasound image review system, such as an ultrasound image review station. In some image review systems, a CPU transfers ultrasound image clips stored in a memory unit to a video display system (e.g. a video display card), which formats the image clips for display on a monitor. Multiple video display systems can be used to display image clips on multiple monitors. In many medical applications, it is important to display the frames of an ultrasound clip at the same rate at which the frames were originally acquired. Furthermore, in special applications such as echocardiography, simultaneous display of multiple cardiac cycles is often required during examination for diagnostic purposes, such that side-by-side comparisons of two or more image sequences or ‘clips’ can be made.

Cycle synchronisation is very important in stress echocardiography, where patient management decisions are made from a visual assessment of the cardiac wall motion and where the digital cycles of digital video sequences are displayed simultaneously for comparison purposes.

Referring to FIG. 7a of the drawings, taking a worst-case example, if one acquired clip (i) contained 10 frames for systole and another (ii) contained 5 frames for systole, systole synchronisation would occur as illustrated in FIG. 7b—i.e. the length of the first clip (i) would first be determined, and then the frames of the second clip (ii) would be distributed equally throughout the length of the first clip by dividing by 2 the frame rate of the second clip (ii), i.e. the two clips are synchronised for a specific process of the anatomical region of interest.

However, simply starting and/or ending two or more different clips (relating to the same process, e.g. systole) of varying lengths at the same time, as in the case illustrated above, is problematic. Playback tends either never to be truly synchronised, or entirely desynchronised. As a result, it becomes difficult to watch all of the clips simultaneously and, in addition, comparisons between clips become very difficult.

### SUMMARY OF THE INVENTION

It is therefore an object of the present invention to provide an improved system for synchronised playback of video image clips.

In accordance with the present invention, there is provided a system for synchronising, for simultaneous display, first and second image clips in respect of an anatomical region of interest of a subject, the system comprising means for receiving data representative of said first and second image clips to be displayed, means for receiving a signal representative of a periodic physiological cycle associated with said anatomical region of interest of said subject, each image clip comprising a set of image frames captured in respect of each of a plurality of respective sequential physiological cycles associated with said anatomical region of interest, means for identifying, in respect of a plurality of physiological cycles, respective sets of image frames associated therewith from said first and second clips, and means for synchronising in respect of each of a plurality of physiological cycles, display of corresponding respective sets of image frames of each of said first and second clips, such that display of each of said first and second clips is synchronised for each respective physiological cycle.

Also in accordance with the present invention, there is provided a method for synchronising, for simultaneous display, first and second image clips in respect of an anatomical region of interest of a subject, the method comprising receiving data representative of said first and second image clips to be displayed, receiving a signal representative of a periodic physiological cycle associated with said anatomical region of interest of said subject, each image clip comprising a set of image frames captured in respect of each of a plurality of respective sequential physiological cycles associated with said anatomical region of interest, identifying, in respect of a plurality of physiological cycles, respective sets of image frames associated therewith from said first and second clips, and synchronising in respect of each of a plurality of physiological cycles, display of corresponding respective sets of image frames of each of said first and second clips, such that display of each of said first and second clips is synchronised for each respective physiological cycle.

The present invention also extends to an image review system comprising display means for displaying the first and second clips, and utilising a system or method as defined above for synchronising, for simultaneous display, the first and second image clips in respect of each of a plurality of respective physiological cycles.

Thus, the present invention provides a mechanism whereby two (or more) image clips in respect of an anatomical region of interest can be synchronised for simultaneous display taking into account the period of the above-mentioned physiological cycles. In a preferred embodiment, the anatomical region of interest may comprise a cardiac region of the subject, and the physiological cycles may comprise cardiac cycles, such that the above-mentioned synchronisation takes into account the heart rate of the subject, as derived from the signal (e.g. an electrocardiograph signal) representative thereof Accordingly, differing frame counts of sets of frames associated with each physiological cycle can be accommodated effectively.

It will be appreciated that the present invention is particularly suited for use in display of ultrasound image clips. However, it will be equally appreciated that the same techniques could be applied for synchronising, for simultaneous display, other types of digital image clips.

In a preferred embodiment, where the number of physiological cycles associated with said first image clip is greater than the number of physiological cycles associated with said second image clip, once all sets of image frames of the second image clip have been displayed synchronised with the corresponding sets of frames of the first image clip, the system may be arranged and configured such that sequential display of the sets of image frames of the second clip is repeated, each set of image frames being synchronised and re-displayed with corresponding next image frame sets of the first image clip. Equally, once all of the sets of image frames of the first image clip have been displayed, sequential display of those sets can be re-started, if desired, such that display of each set of frames of the first clip is synchronised with that of the next sequence of image frame sets of the second image clip to be displayed.

In other words, display of a shorter clip (i.e. one covering less physiological cycles than another clip) can wrap around to the beginning and remain synchronised with the longer clip, while maintaining continuous playback of both clips. The absolute length of each of the image clips becomes irrelevant.

Beneficially, display of the first and second clips is arranged to start and end simultaneously.

The present invention can be applied to synchronising, for simultaneous display, more than two image clips at a time, with the additional advantage that this allows for a complete review page of clips of varying lengths to exhibit synchronised playback.

In one exemplary embodiment of the present invention, synchronisation of two or more sets of frames to be displayed in respect of a particular physiological cycle is achieved by determining the length of each set, identifying the set having the longest length, and adjusting, for output and display, the frame count of the image frames of the other sets so as to fit the length of the longest image frame set.

These and other aspects of the invention will be apparent from and will be elucidated with reference to the embodiments described hereinafter.

### BRIEF DESCRIPTION OF THE DRAWINGS

The present invention will now be described in more detail, by way of example, with reference to the accompanying drawings, wherein:

FIGS. 1a, 1b and 1c illustrate schematically respective image sequences or clips captured during a respective heart cycle;

FIG. 2 illustrates schematically the principle of a portion of a method according to an exemplary embodiment of the present invention of synchronised playback of the image sequences illustrated in FIGS. 1a, 1b and 1c;

FIG. 3 is a schematic diagram illustrating an ultrasound image review system according to an exemplary embodiment of the present invention;

FIG. 4 is a schematic block diagram illustrating principle components of the computer unit of the system of FIG. 3;

FIG. 5 is a block diagram illustrating schematically an exemplary implementation of the video display systems of the arrangement of FIG. 4;

FIG. 6 illustrates schematically the principle of a portion of a method of synchronised playback according to an exemplary embodiment of the present invention;

FIG. 7a illustrates schematically first (i) and second (ii) image clips acquired in respect of a specified process, such as systole, of a patient's anatomical region of interest; and

FIG. 7b illustrates schematically, the manner in which the second image clip (ii) of FIG. 7a is synchronised relative to the first image clip (i) according to the prior art.

### DETAILED DESCRIPTION OF THE INVENTION

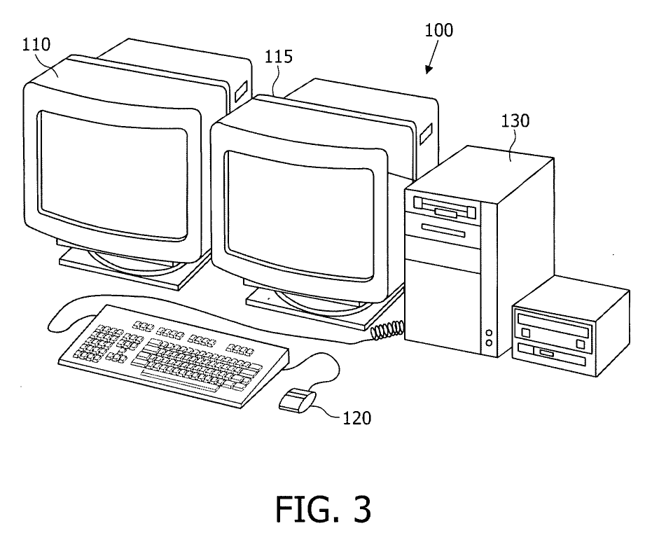

Referring to FIG. 3 of the drawings, there is illustrated schematically an ultrasound image review system 100 according to an exemplary embodiment of the present invention. As used herein, the term “ultrasound image review system” refers to any device that can display digital ultrasound images. Ultrasound image review systems include, but are not limited to, ultrasound image review stations and ultrasound image acquisition devices. The ultrasound image review system 100 of FIG. 3 takes the form of an ultrasound image review station comprising a first and second monitor 110, 115, a mouse 120, and a computer unit 130. Although two monitors are shown in FIG. 3, the ultrasound image review system 100 can have only one monitor or can have three or more monitors.

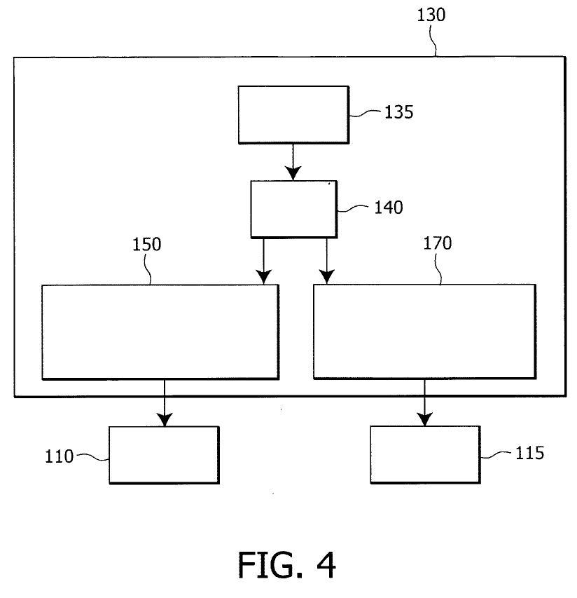

FIG. 4 is a schematic block diagram of an exemplary computer unit 130. Of course, computer 130 may comprise components in addition to the ones shown in FIG. 4. Many, if not all of these components may depend upon the particular computer used and are, therefore, not shown in FIG. 4. In this exemplary embodiment, the computer 130 is a general purpose computer and comprises a memory unit 135 coupled with a CPU 140. As used herein, the term “coupled with” means directly coupled with or indirectly coupled with through one or more components. The CPU 140 is coupled with two video display systems 150, 170 (e.g. video cards), which are coupled with monitors 110, 115 respectively. Additional monitors can be added to the system 100 by adding additional video display systems to the computer unit 130. In this case, the term “video display system” refers to a self-contained system (i.e. independent of the CPU 140 of the image review system 100) that is operative to receive ultrasound data and render, from the ultrasound data, a viewable image on a monitor.

In operation, the review system 100 can be used to review ultrasound image clips that are digitally stored in the memory unit 135. As used herein, the term “ultrasound image clip” refers to a plurality of ultrasound image frames. An ultrasound image clip can be, for example, a series of ultrasound images that are acquired when an ultrasound transducer is swept across a patient. An ultrasound image clip can be transferred to the memory unit 135 from an ultrasound acquisition device via a direct connection between the review system and an acquisition device, or via an indirect connection such as a network. Additionally, ultrasound image clips digitally saved on a portable medium, such as a magneto-optical disc, can be transferred into the memory unit 135 of the system 100.

The video display systems 150, 170 are operative to simultaneously display multiple ultrasound image clips, wherein the rate at which the frames of each ultrasound image clip are displayed (“the display frame rate”) is the same as the rate at which the frames were acquired (“the acquired frame rate”). This will now be described in more detail.

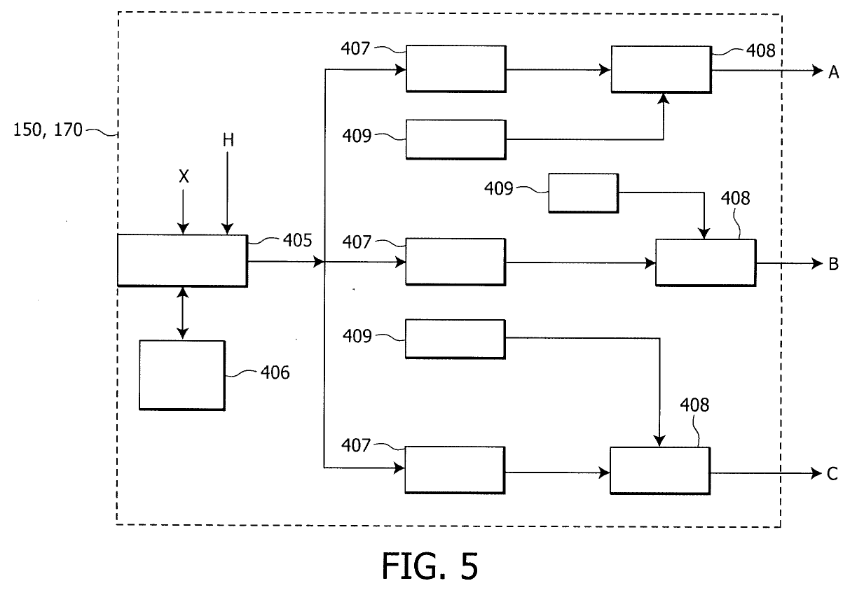

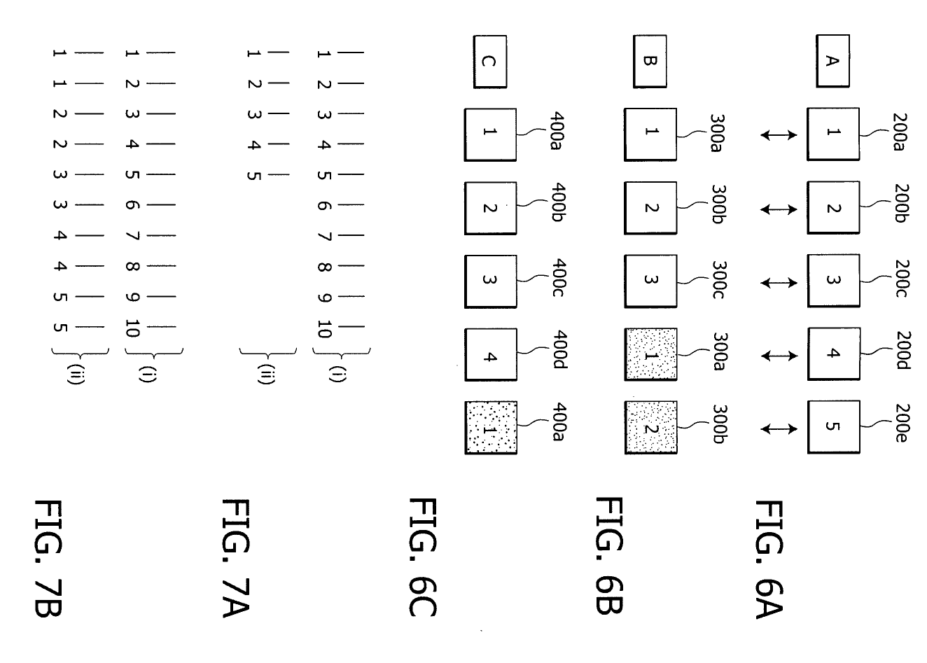

Referring to FIG. 5 of the drawings, there is provided a highly simplified block diagram which illustrates schematically the basic principle of an exemplary implementation of a video display system 150, 170. The video display system 150, 170 may comprise a controller 405 for controlling a plurality of image output arms (in this case three), which output the frames of each clip A, B and C for display on the monitor (FIG. 4—110, 115). Inputs to the controller include X: ultrasound image data, and H: an ECG (echocardiograph) signal representative of each of a plurality of cardiac cycles to which output of the image frames for display is to be synchronised. A memory 406 is coupled to the controller 405.

Each output arm comprises a FIFO (First-in-First-out) register 407 for receiving the frames of a respective clip to be output for display and a processor 408 for synchronising the frame(s) of the clips for each cardiac cycle prior to output thereof for display. Each arm also includes a buffer 409 corresponding to each respective processor 408.

Referring to FIG. 6 of the drawings, by synchronising each currently-viewed cardiac cycle H to each other, the absolute length of each captured image clip becomes irrelevant to the synchronisation process. In this exemplary embodiment of the present invention, synchronisation accommodates both the heart rate and the differing frame counts between the frames of the respective clips for a given cardiac cycle H. Synchronised clips should start and end simultaneously, but in this case, if the second clip covers less cardiac cycles than the first, it simply re-starts so that the frames covering the first cardiac cycle are synchronised with the frames of the next cycle of the first clip.

For example, as illustrated in FIG. 6a of the drawings, clip A may cover 5 cardiac cycles 200a, b, . . . , e whereas clip B may only cover 3 cardiac cycles 300a, b, c, as shown in FIG. 6b. In this case, synchronisation of clips A and B would occur as follows. The first cycle 200a of Clip A is synchronised with cycle 300a of clip B. Cycle 200b of clip A synchronises with cycle 300b of clip B, and cycle 200c of clip A synchronises with cycle 300c of clip B. At this point, clip B has reached its end. Thus, to maintain continuous playback of both clips, clip B will re-start at the first cycle 300a, while playback of clip A continues with the fourth cycle 200d, and these two cycles are synchronised as before. This process can, in theory at least, be continued ad infinitum, or until the user pauses or stops playback.

In summary, the present invention provides a system in which each clip specific to a currently-viewed cardiac cycle is synchronised. A shorter clip can wrap around to the beginning and remain synchronised to the longer clip while maintaining continuous playback of both clips. Thus, the synchronized clips start and end simultaneously. Further, this process can be applied to more than two clips at a time, thereby allowing a complete review page of clips of varying lengths to exhibit synchronised playback.

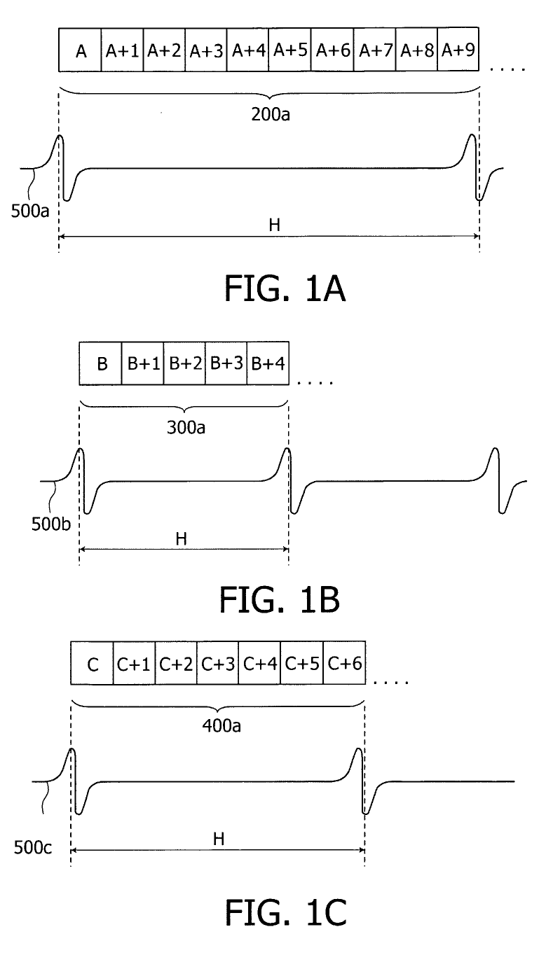

Referring to FIG. 1a of the drawings, the ultrasonic cardiac images A, A+1, A+2, . . . , A+9 captured during a first cycle 200a of sequence A are illustrated schematically. Referring to FIG. 1b of the drawings, the ultrasonic cardiac images B, B+1, . . . , B+4 captured during a first cycle 300a of sequence B are illustrated schematically. Referring to FIG. 1c of the drawings, the ultrasonic cardiac images C, C+1, C+2, . . . , C+6 captured during a first cycle 400a of sequence C are illustrated schematically. Generally, the ultrasound system concurrently monitors the heart cycle with an ECG electrode, and the resultant ECG waveform 500 is concurrently displayed along with the cardiac images. All three illustrated sequences were acquired in realtime, and relate to a single heart cycle, H, as defined by the corresponding respective waveform 500a, b and c.

An exemplary manner in which the corresponding portions of each sequence relating to the respective cardiac cycle could be synchronised will now be described in more detail. However, it will be appreciated that other methods of synchronisation are envisaged, and the present invention is not intended to be limited in this regard.

Referring to FIG. 1 of the drawings, the cycles 200a, 300a and 400a of sequences A, B and C illustrated in FIGS. 1a, 1b and 1c respectively can be considered to have been captured at (the same) video frame rate. Therefore, the illustrated cycles of sequences (or ‘clips’) A, B and C each contain a different number of frames, i.e. in this case, 10, 5 and 7 respectively, and are therefore of varying lengths.

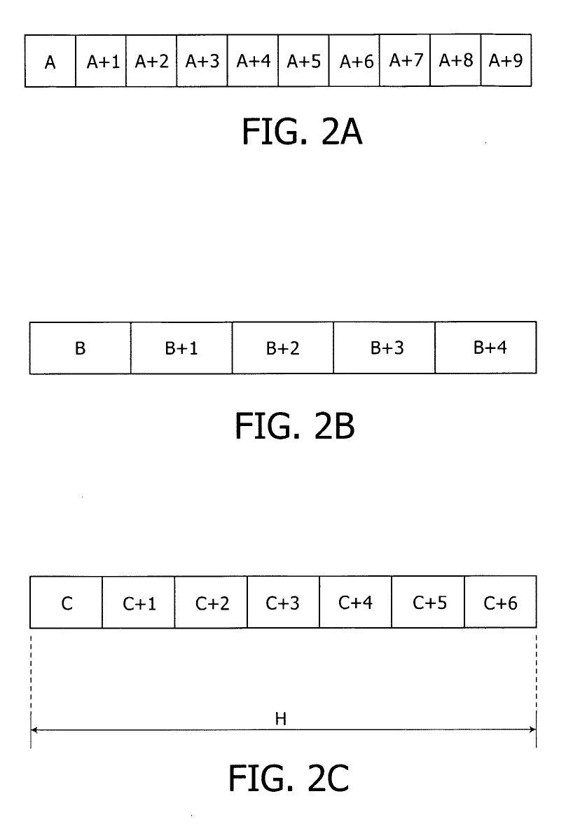

In the illustrated example, synchronisation of each cycle H may occur as illustrated schematically in FIG. 2. It can be seen that the cycle 200a (sequence A) having the largest number of frames (captured during the period of slowest heart rate) is used as the basis for synchronisation. The frames of cycle 200a are played back at the same speed as that of acquisition thereof. Cycle 300a has half the number of frames of cycle 200a, and playback of cycle 300a therefore takes place at half the speed of acquisition, such that playback of cycles 200a and 300a starts and ends at the same point. Cycle 400a has 70% of the number of frames of cycle 200a, and it is played back at 70% of the speed of acquisition such that, once again, playback of cycles 200a and 400a starts and ends at the same point.

However, it will be appreciated that other methods of synchronisation of the corresponding portions of each sequence may be employed in respect of the present invention, provided each of said portions corresponds to a predetermined cycle of the anatomical region of interest, i.e. in this case, the cardiac cycle.

It should be noted that the above-mentioned embodiment illustrates rather than limits the invention, and that those skilled in the art will be capable of designing many alternative embodiments without departing from the scope of the invention as defined by the appended claims. In the claims, any reference signs placed in parentheses shall not be construed as limiting the claims. The word “comprising” and “comprises”, and the like, does not exclude the presence of elements or steps other than those listed in any claim or the specification as a whole. The singular reference of an element does not exclude the plural reference of such elements and vice-versa. The invention may be implemented by means of hardware comprising several distinct elements, and by means of a suitably programmed computer. In a device claim enumerating several means, several of these means may be embodied by one and the same item of hardware. The mere fact that certain measures are recited in mutually different dependent claims does not indicate that a combination of these measures cannot be used to advantage.

1. A system for synchronising, for simultaneous display, first and second image clips in respect of an anatomical region of interest of a subject, the system comprising means for receiving data representative of said first and second image clips to be displayed, means for receiving a signal representative of a periodic physiological cycle associated with said anatomical region of interest of said subject, each image clip comprising a set of image frames captured in respect of each of a plurality of respective sequential physiological cycles associated with said anatomical region of interest, means for identifying, in respect of a plurality of physiological cycles, respective sets of image frames associated therewith from said first and second clips, and means for synchronising in respect of each of a plurality of physiological cycles, display of corresponding respective sets of image frames of each of said first and second clips, such that display of each of said first and second clips is synchronised for each respective physiological cycle.

2. A system according to claim 1, arranged and configured such that, in the case that the number of physiological cycles associated with said first image clip is greater than the number of physiological cycles associated with said second image clip, once all sets of image frames of the second image clip have been displayed synchronised with the corresponding sets of frames of the first image clip sequential display of the sets of image frames of the second clip is repeated, each set (of image frames being synchronised and re-displayed with corresponding next image frame sets of the first image clip.

3. A system according to claim 2, wherein once all of the sets of image frames of the first image clip have been displayed, sequential display of those sets can be re-started, if desired, such that display of each set of frames of the first clip is synchronised with that of the next sequence of image frame sets of the second image clip to be displayed.

4. A system according to claim 1, wherein display of the first and second clips is arranged to start and end simultaneously.

5. A system according to claim 1, wherein the anatomical region of interest comprises a cardiac region of the subject, and the physiological cycles comprise cardiac cycles.

6. A system according to claim 5, wherein said signal representative of said physiological cycles comprises an ECG signal.

7. A system according to claim 1, wherein said image clips comprise ultrasound image clips.

8. A system according to claim 1, wherein synchronisation of two or more sets of frames to be displayed in respect of a particular physiological cycle is achieved by determining the length of each set, identifying the set having the longest length, and adjusting, for output and display, the frame count of the image frames of the other sets so as to fit the length of the longest image frame set.

9. A method for synchronising, for simultaneous display, first and second image clips in respect of an anatomical region of interest of a subject, the method comprising receiving data representative of said first and second image clips to be displayed, receiving a signal representative of a periodic physiological cycle associated with said anatomical region of interest of said subject, each image clip comprising a set of image frames captured in respect of each of a plurality of respective sequential physiological cycles associated with said anatomical region of interest, identifying, in respect of a plurality of physiological cycles, respective sets of image frames associated therewith from said first and second clips, and synchronising in respect of each of a plurality of physiological cycles, display of corresponding respective sets of image frames of each of said first and second clips, such that display of each of said first and second clips is synchronised for each respective physiological cycle.

10. An image review system comprising display means for displaying first and second image clips and a system according to claim 1 for synchronising, for simultaneous display, the first and second image clips in respect of each of a plurality of respective physiological cycles.
